(19) United States
(12) Patent Application Publication
(10) Pub. No.: US 2025/0137062 A1
(43) Pub. Date: May 1, 2025

(54) FGFR1 FUSIONS IN OSTEOSARCOMA AND METHODS AND USES RELATED THERETO

(71) Applicant: Foundation Medicine, Inc., Boston, MA (US)

(72) Inventors: Zheng KUANG, Boston, MA (US); Matthew C. HIEMENZ, Chestnut Hill, MA (US)

(73) Assignee: Foundation Medicine, Inc., Boston, MA (US)

(21) Appl. No.: 18/930,614

(22) Filed: Oct. 29, 2024

Related U.S. Application Data
(60) Provisional application No. 63/546,422, filed on Oct. 30, 2023.

(51) Int. Cl. C12Q 1/6886

(52) U.S. Cl. CPC C12Q 1/6886; C12Q 2600/112; C12Q 2600/156

(57) ABSTRACT

Provided herein are methods related to treating or delaying progression of osteosarcoma in an individual. In some embodiments, the methods comprise detecting an FGFR1-MTSS1 or TACC1-FGFR1 gene fusion in one or more samples from an individual having a cancer; and administering to the individual an effective amount of a treatment that comprises an anti-cancer agent.

### CROSS-REFERENCE TO RELATED APPLICATIONS

This application claims the benefit of U.S. Provisional Application No. 63/546,422, filed Oct. 30, 2023, which is hereby incorporated by reference in its entirety.

### REFERENCE TO AN ELECTRONIC SEQUENCE LISTING

The contents of the electronic sequence listing (197102016400seglist.xml; Size: 13,449 bytes; and Date of Creation: Oct. 28, 2024) are incorporated herein by reference in their entirety.

### TECHNICAL FIELD

Provided herein are methods related to treating or delaying progression of osteosarcoma in an individual. In some embodiments, the methods comprise detecting an FGFR1-MTSS1 or TACC1-FGFR1 gene fusion in one or more samples from an individual having a cancer; and administering to the individual an effective amount of a treatment that comprises an anti-cancer agent.

### BACKGROUND

Osteosarcoma is the most common primary bone cancer in children and young adults; however, it remains a rare cancer, with an incidence of roughly 4 cases per 1 million patients <40 years old in the US [1]. Incidence of osteosarcoma follows a bimodal distribution, occurring most frequently in adolescents, with a second peak among individuals aged >60 years [2, 3]. The 5-year survival rate is approximately 60% among patients with localized osteosarcoma but is only around 20% among patients presenting with metastatic or recurrent disease [4]. Unfortunately, survival rates have not significantly improved since the introduction of modern chemotherapy regimens in the 1980s.

Various large-scale studies have been undertaken to help characterize the genomic characteristics of osteosarcoma [5-14]. The osteosarcoma genome is characterized by widespread structural rearrangements and clustered mutational processes with evidence of chromothripsis, chromoplexy and kataegis in most cases [5, 15]. These processes lead to a high burden of structural variants (SVs) and copy number variants (CNVs) and a relatively low burden of mutations which are typically clustered at breakpoints. Relatively few recurrent gene mutations are identified. Among the genes with recurrent mutations are the tumor suppressors TP53 and RB1, through rearrangements or deletions are the more common mechanisms of loss for these genes [5, 6, 11]. Recurrent CNVs include MYC, CCNE1, and CDK4 amplifications, and CDKN2A/B, TP53 and RB1 deletions [9].

While improved understanding of osteosarcoma biology may provide hope for innovative targeted therapy options, recent advances in cancer care like precision oncology and immunotherapy are not effective in osteosarcoma [16]. Biologic subtypes associated with treatment response and resistance have not been described in osteosarcoma. Recently, MYC amplification has been demonstrated to be associated with worse outcome in osteosarcoma providing proof of principle for biologic subtypes [14]. Genomic complexity and small case numbers have made statistically robust assessment of mutational patterns difficult in prior studies and have likely prevented identification of biologic subtypes.

### SUMMARY OF THE INVENTION

In one aspect, provided herein is a method of treating or delaying progression of osteosarcoma in an individual, comprising detecting an FGFR1-MTSS1 or TACC1-FGFR1 gene fusion in one or more samples from an individual having osteosarcoma; and administering to the individual an effective amount of a treatment that comprises an anti-cancer agent.

In some embodiments, the anti-cancer agent comprises an FGFR1 inhibitor, tyrosine kinase inhibitor, or pan-kinase inhibitor. In some embodiments, the FGFR1-MTSS1 gene fusion encodes a fusion polypeptide comprising the amino acid sequence of MWSWKCLLFWAVLVTATLCTARPSPTLPEQAQPWGAPVEVESFLVHPGDLLQLRCRLRDDV QSINWLRDGVQLAESNRTRITGEEVEVQDSVPADSGLYACVTSSPSGSDTTYFSVNVSDALPS SEDDDDDDDSSSEEKETDNTKPNPVAPYWTSPEKMEKKLHAVPAAKTVKFKCPSSGTPNPTL RWLKNGKEFKPDHRIGGYKVRYATWSIIMDSVVPSDKGNYTCIVENEYGSINHTYQLDVVER SPHRPILQAGLPANKTVALGSNVEFMCKVYSDPQPHIQWLKHIEVNGSKIGPDNLPYVQILKT AGVNTTDKEMEVLHLRNVSFEDAGEYTCLAGNSIGLSHHSAWLTVLEALEERPAVMTSPLY LEIIIYCTGAFLISCMVGSVIVYKMKSGTKKSDFHSQMAVHKLAKSIPLRRQVTVSADSSASM NSGVLLVRPSRLSSSGTPMLAGVSEYELPEDPRWELPRDRLVLGKPLGEGCFGQVVLAEAIGL DKDKPNRVTKVAVKMLKSDATEKDLSDLISEMEMMKMIGKHKNIINLLGACTQDGPLYVIV EYASKGNLREYLQARRPPGLEYCYNPSHNPEEQLSSKDLVSCAYQVARGMEYLASKKCIHR DLAARNVLVTEDNVMKIADFGLARDIHHIDYYKKTTNGRLPVKWMAPEALFDRIYTHQSDV WSFGVLLWEIFTLGGSPYPGVPVEELFKLLKEGHRMDKPSNCTNELYMMMRDCWHAVPSQ RPTFKQLVEDLDRIVALTSNQDNSSSSSCLLGRLSESG (SEQ ID NO:1). In some embodiments, the FGFR1-MTSS1 gene fusion comprises the polynucleotide sequence of ATGTGGAGCTGGAAGTGCCTCCTCTTCTGGGCTGTGCTGGTCACAGCCACACTCTGCACC GCTAGGCCGTCCCCGACCTTGCCTGAACAAGCCCAGCCCTGGGGAGCCCCTGTGGAAGT GGAGTCCTTCCTGGTCCACCCCGGTGACCTGCTGCAGCTTCGCTGTCGGCTGCGGGACGA TGTGCAGAGCATCAACTGGCTGCGGGACGGGGTGCAGCTGGCGGAAAGCAACCGCACCC GCATCACAGGGGAGGAGGTGGAGGTGCAGGACTCCGTGCCCGCAGACTCCGGCCTCTAT GCTTGCGTAACCAGCAGCCCCTCGGGCAGTGACACCACCTACTTCTCCGTCAATGTTTCA GATGCTCTCCCCTCCTCGGAGGATGATGATGATGATGATGACTCCTCTTCAGAGGAGAAA GAAACAGATAACACCAAACCAAACCCCGTAGCTCCATATTGGACATCCCCAGAAAAGAT GGAAAAGAAATTGCATGCAGTGCCGGCTGCCAAGACAGTGAAGTTCAAATGCCCTTCCA GTGGGACCCCAAACCCCACACTGCGCTGGTTGAAAAATGGCAAAGAATTCAAACCTGAC CACAGAATTGGAGGCTACAAGGTCCGTTATGCCACCTGGAGCATCATAATGGACTCTGTG GTGCCCTCTGACAAGGGCAACTACACCTGCATTGTGGAGAATGAGTACGGCAGCATCAA CCACACATACCAGCTGGATGTCGTGGAGCGGTCCCCTCACCGGCCCATCCTGCAAGCAG GGTTGCCCGCCAACAAAACAGTGGCCCTGGGTAGCAACGTGGAGTTCATGTGTAAGGTG TACAGTGACCCGCAGCCGCACATCCAGTGGCTAAAGCACATCGAGGTGAATGGGAGCAA GATTGGCCCAGACAACCTGCCTTATGTCCAGATCTTGAAGACTGCTGGAGTTAATACCAC CGACAAAGAGATGGAGGTGCTTCACTTAAGAAATGTCTCCTTTGAGGACGCAGGGGAGT ATACGTGCTTGGCGGGTAACTCTATCGGACTCTCCCATCACTCTGCATGGTTGACCGTTCT GGAAGCCCTGGAAGAGAGGCCGGCAGTGATGACCTCGCCCCTGTACCTGGAGATCATCA TCTATTGCACAGGGGCCTTCCTCATCTCCTGCATGGTGGGGTCGGTCATCGTCTACAAGA TGAAGAGTGGTACCAAGAAGAGTGACTTCCACAGCCAGATGGCTGTGCACAAGCTGGCC AAGAGCATCCCTCTGCGCAGACAGGTAACAGTGTCTGCTGACTCCAGTGCATCCATGAAC TCTGGGGTTCTTCTGGTTCGGCCATCACGGCTCTCCTCCAGTGGGACTCCCATGCTAGCA GGGGTCTCTGAGTATGAGCTTCCCGAAGACCCTCGCTGGGAGCTGCCTCGGGACAGACT GGTCTTAGGCAAACCCCTGGGAGAGGGCTGCTTTGGGCAGGTGGTGTTGGCAGAGGCTA TCGGGCTGGACAAGGACAAACCCAACCGTGTGACCAAAGTGGCTGTGAAGATGTTGAAG TCGGACGCAACAGAGAAAGACTTGTCAGACCTGATCTCAGAAATGGAGATGATGAAGAT GATCGGGAAGCATAAGAATATCATCAACCTGCTGGGGGCCTGCACGCAGGATGGTCCCT TGTATGTCATCGTGGAGTATGCCTCCAAGGGCAACCTGCGGGAGTACCTGCAGGCCCGG AGGCCCCCAGGGCTGGAATACTGCTACAACCCCAGCCACAACCCAGAGGAGCAGCTCTC CTCCAAGGACCTGGTGTCCTGCGCCTACCAGGTGGCCCGAGGCATGGAGTATCTGGCCTC CAAGAAGTGCATACACCGAGACCTGGCAGCCAGGAATGTCCTGGTGACAGAGGACAATG TGATGAAGATAGCAGACTTTGGCCTCGCACGGGACATTCACCACATCGACTACTATAAA AAGACAACCAACGGCCGACTGCCTGTGAAGTGGATGGCACCCGAGGCATTATTTGACCG GATCTACACCCACCAGAGTGATGTGTGGTCTTTCGGGGTGCTCCTGTGGGAGATCTTCAC TCTGGGCGGCTCCCCATACCCCGGTGTGCCTGTGGAGGAACTTTTCAAGCTGCTGAAGGA GGGTCACCGCATGGACAAGCCCAGTAACTGCACCAACGAGCTGTACATGATGATGCGGG ACTGCTGGCATGCAGTGCCCTCACAGAGACCCACCTTCAAGCAGCTGGTGGAAGACCTG GACCGCATCGTGGCCTTGACCTCCAACCAGGACTGTATTTTGAGCCTGAACCAATTTCTT CCACGCCCAATTATTTGCAACGGGGAGAATTTTCCAGTTGTGTTTCATGTGAAGAAAACT CAAGCTGCCTCGACCAGATCTTTGATTCCTACCTTCAGACAGAGGACAACAGTAGTAGCA GCAGCTGCCTTCTTGGACGCCTTTCAGAAAGTGGCTGACATGGCCACCAACACACGTGGT GGGACCAGGGAGATTGGATCTGCTCTCACCAGGATGTGCATGAGGCACAGAAGCATTGA AGCCAAGCTGAGGCAGTTTTCGAGCGCTTTAATTGATTGTCTGATAAACCCACTTCAAGA ACAGATGGAAGAATGGAAGAAAGTGGCCAACCAGCTGGATAAAGACCACGCAAAAGAA TATAAGAAAGCCCGCCAAGAGATAAAAAAGAAGTCCTCGGATACGCTGAAACTGCAGA AGAAAGCAAAAAAAGGGAGAGGTGATATCCAGCCTCAGTTGGACAGTGCTCTCCAAGAT GTCAATGATAAGTATCTCTTATTGGAAGAAACAGAAAAGCAGGCTGTCCGGAAGGCTTT GATTGAAGAACGTGGCCGATTCTGTACCTTCATCTCTATGCTGCGGCCAGTGATTGAAGA AGAAATCTCAATGCTAGGGGAAATAACCCACCTTCAGACCATCTCGGAAGATCTAAAAA GCCTGACCATGGACCCTCACAAACTGCCCTCCTCAAGTGAACAGGTGATTCTGGACTTGA AAGGTTCTGATTACAGCTGGTCGTATCAGACGCCACCCTCTTCCCCCAGCACCACCATGT CCAGAAAGTCCAGTGTCTGCAGCAGCCTGAACAGTGTCAACAGCAGTGACTCCCGGTCC AGCGGCTCCCACTCGCATTCCCCCAGCTCACATTACCGCTACCGCAGCTCCAACCTGGCC CAGCAGGCTCCTGTGAGGCTGTCCAGCGTGTCCTCCCATGACTCAGGATTCATATCCCAG GATGCCTTCCAGTCCAAGTCACCATCCCCCATGCCGCCAGAGGCCCCCAACCAGTTGTCT AACGGGTTTTCTCACTATAGTTTATCAAGTGAGTCCCACGTGGGGCCCACGGGTGCAGGC CTTTTCCCTCATTGCCTGCCTGCCTCCCGCCTGCTCCCTCGGGTCACCTCTGTCCACCTTCC AGACTACGCTCATTATTACACCATTGGGCCCGGCATGTTCCCGTCATCTCAGATCCCTAG CTGGAAGGACTGGGCTAAGCCTGGGCCCTATGACCAGCCTCTGGTGAACACCCTGCAGC GCCGCAAAGAGAAGCGAGAACCGGACCCCAACGGGGGAGGACCCACTACCGCCAGCGG CCCACCTGCAGCAGCTGAGGAGGCTCAGAGACCACGGAGCATGACTGTATCGGCTGCCA CCAGGCCTGGTGAGGAGATGGAGGCTTGTGAGGAGCTGGCCCTGGCCCTGTCTCGGGGC CTGCAGCTGGACACCCAGAGGAGCAGCCGGGACTCGCTTCAGTGCTCCAGCGGCTACAG CACCCAGACAACCACCCCCTGCTGCTCTGAGGACACCATCCCTTCCCAAGTTTCAGATTA TGATTATTTCTCTGTAAGTGGTGACCAGGAGGCAGATCAGCAGGAGTTCGACAAGTCCTC CACCATTCCAAGAAACAGCGACATCAGCCAGTCCTACCGACGGATGTTCCAAGCCAAGC GTCCAGCCTCAACTGCTGGCCTCCCCACCACCCTGGGACCTGCTATGGTCACTCCAGGGG TTGCAACTATCCGACGGACCCCTTCCACCAAGCCTTCTGTCCGCCGGGGAACCATTGGAG CTGGTCCCATCCCCATCAAGACACCCGTGATCCCTGTCAAGACCCCAACCGTCCCAGACC TCCCAGGGGTGTTGCCAGCCCCTCCAGATGGGCCAGAAGAGCGGGGGGAGCACAGCCCT GAGTCGCCATCTGTGGGTGAGGGCCCCCAAGGTGTCACCAGCATGCCCTCCTCAATGTGG AGCGGCCAAGCTTCCGTTAACCCTCCACTTCCAGGCCCGAAGCCCAGTATCCCTGAGGAG CACAGACAGGCAATTCCAGAAAGTGAAGCTGAAGACCAGGAACGGGAACCCCCAAGTG CCACTGTCTCCCCAGGCCAGATTCCAGAGAGTGACCCTGCAGACCTGAGCCCAAGGGAT ACTCCACAAGGAGAAGACATGCTGAACGCCATCCGAAGGGGCGTGAAACTGAAGAAGA CCACGACAAACGATCGCTCAGCCCCTCGCTTTTCTTAG (SEQ ID NO:2). In some embodiments, the FGFR1-MTSS1 gene fusion comprises a fusion junction between exon 17 of FGFR1 and exon 2 of MTSS1. In some embodiments, the FGFR1-MTSS1 gene fusion comprises exons 1-17 of FGFR1 and exons 2-14 of MTSS1 (e.g., from N-terminus to C-terminus).

In some embodiments, the TACC1-FGFR1 gene fusion encodes a fusion polypeptide comprising the amino acid sequence of MAFSPWQILSPVQWAKWTWSAVRGGAAGEDEAGGPEGDPEEEDSQAETKSLSFMWSWKCL LFWAVLVTATLCTARPSPTLPEQAQPWGAPVEVESFLVHPGDLLQLRCRLRDDVQSINWLRD GVQLAESNRTRITGEEVEVQDSVPADSGLYACVTSSPSGSDTTYFSVNVSDALPSSEDDDDDD DSSSEEKETDNTKPNPVAPYWTSPEKMEKKLHAVPAAKTVKFKCPSSGTPNPTLRWLKNGK EFKPDHRIGGYKVRYATWSIIMDSVVPSDKGNYTCIVENEYGSINHTYQLDVVERSPHRPILQ AGLPANKTVALGSNVEFMCKVYSDPQPHIQWLKHIEVNGSKIGPDNLPYVQILKTAGVNTTD KEMEVLHLRNVSFEDAGEYTCLAGNSIGLSHHSAWLTVLEALEERPAVMTSPLYLEIIIYCTG AFLISCMVGSVIVYKMKSGTKKSDFHSQMAVHKLAKSIPLRRQVTVSADSSASMNSGVLLVR PSRLSSSGTPMLAGVSEYELPEDPRWELPRDRLVLGKPLGEGCFGQVVLAEAIGLDKDKPNR VTKVAVKMLKSDATEKDLSDLISEMEMMKMIGKHKNIINLLGACTQDGPLYVIVEYASKGN LREYLQARRPPGLEYCYNPSHNPEEQLSSKDLVSCAYQVARGMEYLASKKCIHRDLAARNV LVTEDNVMKIADFGLARDIHHIDYYKKTTNGRLPVKWMAPEALFDRIYTHQSDVWSFGVLL WEIFTLGGSPYPGVPVEELFKLLKEGHRMDKPSNCTNELYMMMRDCWHAVPSQRPTFKQLV EDLDRIVALTSNQEYLDLSMPLDQYSPSFPDTRSSTCSSGEDSVFSHEPLPEEPCLPRHPAQLA NGGLKRR (SEQ ID NO:3). In some embodiments, the TACC1-FGFR1 gene fusion comprises the polynucleotide sequence of ATGGCGTTCAGCCCGTGGCAGATCCTGTCCCCCGTGCAGTGGGCGAAATGGACGTGGTCT GCGGTACGCGGCGGGGCCGCCGGCGAGGACGAGGCTGGCGGGCCCGAGGGCGACCCCG AGGAGGAGGATTCGCAAGCCGAGACCAAATCCTTGAGTTTCAGGACTGTATTTTGAGCCT GAACCAATTTCTTCCACGCCCAATTATTTGCAACGGGGAGAATTTTCCAGTTGTGTTTCAT GTGAAGAAAACTCAAGCTGCCTCGACCAGATCTTTGATTCCTACCTTCAGACAGAGATGT GGAGCTGGAAGTGCCTCCTCTTCTGGGCTGTGCTGGTCACAGCCACACTCTGCACCGCTA GGCCGTCCCCGACCTTGCCTGAACAAGCCCAGCCCTGGGGAGCCCCTGTGGAAGTGGAG TCCTTCCTGGTCCACCCCGGTGACCTGCTGCAGCTTCGCTGTCGGCTGCGGGACGATGTG CAGAGCATCAACTGGCTGCGGGACGGGGTGCAGCTGGCGGAAAGCAACCGCACCCGCAT CACAGGGGAGGAGGTGGAGGTGCAGGACTCCGTGCCCGCAGACTCCGGCCTCTATGCTT GCGTAACCAGCAGCCCCTCGGGCAGTGACACCACCTACTTCTCCGTCAATGTTTCAGATG CTCTCCCCTCCTCGGAGGATGATGATGATGATGATGACTCCTCTTCAGAGGAGAAAGAAA CAGATAACACCAAACCAAACCCCGTAGCTCCATATTGGACATCCCCAGAAAAGATGGAA AAGAAATTGCATGCAGTGCCGGCTGCCAAGACAGTGAAGTTCAAATGCCCTTCCAGTGG GACCCCAAACCCCACACTGCGCTGGTTGAAAAATGGCAAAGAATTCAAACCTGACCACA GAATTGGAGGCTACAAGGTCCGTTATGCCACCTGGAGCATCATAATGGACTCTGTGGTGC CCTCTGACAAGGGCAACTACACCTGCATTGTGGAGAATGAGTACGGCAGCATCAACCAC ACATACCAGCTGGATGTCGTGGAGCGGTCCCCTCACCGGCCCATCCTGCAAGCAGGGTTG CCCGCCAACAAAACAGTGGCCCTGGGTAGCAACGTGGAGTTCATGTGTAAGGTGTACAG TGACCCGCAGCCGCACATCCAGTGGCTAAAGCACATCGAGGTGAATGGGAGCAAGATTG GCCCAGACAACCTGCCTTATGTCCAGATCTTGAAGACTGCTGGAGTTAATACCACCGACA AAGAGATGGAGGTGCTTCACTTAAGAAATGTCTCCTTTGAGGACGCAGGGGAGTATACG TGCTTGGCGGGTAACTCTATCGGACTCTCCCATCACTCTGCATGGTTGACCGTTCTGGAA GCCCTGGAAGAGAGGCCGGCAGTGATGACCTCGCCCCTGTACCTGGAGATCATCATCTAT TGCACAGGGGCCTTCCTCATCTCCTGCATGGTGGGGTCGGTCATCGTCTACAAGATGAAG AGTGGTACCAAGAAGAGTGACTTCCACAGCCAGATGGCTGTGCACAAGCTGGCCAAGAG CATCCCTCTGCGCAGACAGGTAACAGTGTCTGCTGACTCCAGTGCATCCATGAACTCTGG GGTTCTTCTGGTTCGGCCATCACGGCTCTCCTCCAGTGGGACTCCCATGCTAGCAGGGGT CTCTGAGTATGAGCTTCCCGAAGACCCTCGCTGGGAGCTGCCTCGGGACAGACTGGTCTT AGGCAAACCCCTGGGAGAGGGCTGCTTTGGGCAGGTGGTGTTGGCAGAGGCTATCGGGC TGGACAAGGACAAACCCAACCGTGTGACCAAAGTGGCTGTGAAGATGTTGAAGTCGGAC GCAACAGAGAAAGACTTGTCAGACCTGATCTCAGAAATGGAGATGATGAAGATGATCGG GAAGCATAAGAATATCATCAACCTGCTGGGGGCCTGCACGCAGGATGGTCCCTTGTATGT CATCGTGGAGTATGCCTCCAAGGGCAACCTGCGGGAGTACCTGCAGGCCCGGAGGCCCC CAGGGCTGGAATACTGCTACAACCCCAGCCACAACCCAGAGGAGCAGCTCTCCTCCAAG GACCTGGTGTCCTGCGCCTACCAGGTGGCCCGAGGCATGGAGTATCTGGCCTCCAAGAA GTGCATACACCGAGACCTGGCAGCCAGGAATGTCCTGGTGACAGAGGACAATGTGATGA AGATAGCAGACTTTGGCCTCGCACGGGACATTCACCACATCGACTACTATAAAAAGACA ACCAACGGCCGACTGCCTGTGAAGTGGATGGCACCCGAGGCATTATTTGACCGGATCTA CACCCACCAGAGTGATGTGTGGTCTTTCGGGGTGCTCCTGTGGGAGATCTTCACTCTGGG CGGCTCCCCATACCCCGGTGTGCCTGTGGAGGAACTTTTCAAGCTGCTGAAGGAGGGTCA CCGCATGGACAAGCCCAGTAACTGCACCAACGAGCTGTACATGATGATGCGGGACTGCT GGCATGCAGTGCCCTCACAGAGACCCACCTTCAAGCAGCTGGTGGAAGACCTGGACCGC ATCGTGGCCTTGACCTCCAACCAGGAGTACCTGGACCTGTCCATGCCCCTGGACCAGTAC TCCCCCAGCTTTCCCGACACCCGGAGCTCTACGTGCTCCTCAGGGGAGGATTCCGTCTTC TCTCATGAGCCGCTGCCCGAGGAGCCCTGCCTGCCCCGACACCCAGCCCAGCTTGCCAAT GGCGGACTCAAACGCCGCTGA (SEQ ID NO:4). In some embodiments, the TACC1-FGFR1 gene fusion comprises a fusion junction between exon 1 of TACC1 and exon 2 of FGFR1. In some embodiments, the TACC1-FGFR1 gene fusion comprises exon 1 of TACC1 and exons 2-17 of FGFR1 (e.g., from N-terminus to C-terminus).

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 shows prevalence of mutations in the indicated genes in osteosarcoma compared to other cancers (pan-cancer).

FIG. 2 shows genes that were significantly enriched (p<0.0001) for alterations in OST relative to other cancers, for example, NCOR1 (log 2(OR)=2.9), IGF1R (log 2(OR)=2.3), FRS2 (log 2(OR)=2.3), and BCL2L2 (log 2(OR)=3.2). The plot shows−log 10(P-value) vs. odds ratio for each indicated gene. Acronyms: OST: osteosarcoma; OR: odds ratio.

FIG. 3 shows the type of variant observed for each indicated gene in the osteosarcoma cohort.

FIG. 4 shows co-occurrence and mutual exclusivity of mutations in indicated genes that were identified with statistical robustness. * indicates p-value<0.05. ** indicates p-value<0.01. *** indicates p-value<1e-4. P-values were derived from fisher-exact test. P-values are FDR-adjusted.

FIG. 5 shows frequency of certain gene alterations varied by age group. Shown in FIG. 5 is a graph of the percentages of child (left bar for each gene), adolescent (middle bar for each gene), and young adult (right bar for each gene) patients with alterations in certain genes. * indicates p-value<0.05. ** indicates p-value<0.01. *** indicates p-value<0.001 (p-values from chi-squared test).

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: FGFR1 Fusions in Osteosarcoma

### INTRODUCTION

Osteosarcoma is the most common primary bone cancer in children and young adults; however, it remains a rare cancer, with an incidence of roughly 4 cases per 1 million patients <40 years old in the US [1]. Incidence of osteosarcoma follows a bimodal distribution, occurring most frequently in adolescents, with a second peak among individuals aged >60 years [2, 3]. The 5-year survival rate is approximately 60% among patients with localized osteosarcoma but is only around 20% among patients presenting with metastatic or recurrent disease [4]. Unfortunately, survival rates have not significantly improved since the introduction of modern chemotherapy regimens in the 1980s.

Various large-scale studies have been undertaken to help characterize the genomic characteristics of osteosarcoma [5-14]. The osteosarcoma genome is characterized by widespread structural rearrangements and clustered mutational processes with evidence of chromothripsis, chromoplexy and kataegis in most cases [5, 15]. These processes lead to a high burden of structural variants (SVs) and copy number variants (CNVs) and a relatively low burden of mutations which are typically clustered at breakpoints. Relatively few recurrent gene mutations are identified. Among the genes with recurrent mutations are the tumor suppressors TP53 and RB1, through rearrangements or deletions are the more common mechanisms of loss for these genes [5, 6, 11]. Recurrent CNVs include MYC, CCNE1, and CDK4 amplifications, and CDKN2A/B, TP53 and RB1 deletions [9].

While improved understanding of osteosarcoma biology may provide hope for innovative targeted therapy options, recent advances in cancer care like precision oncology and immunotherapy are not effective in osteosarcoma [16]. Biologic subtypes associated with treatment response and resistance have not been described in osteosarcoma. Recently, MYC amplification has been demonstrated to be associated with worse outcome in osteosarcoma providing proof of principle for biologic subtypes [14]. Genomic complexity and small case numbers have made statistically robust assessment of mutational patterns difficult in prior studies and have likely prevented identification of biologic subtypes.

In this study, frequently altered genes in 657 pediatric, adolescent, and young adult (P-AYA) patients with osteosarcoma who underwent targeted tumor sequencing were analyzed. To date, this represents the largest reported study of sequencing on osteosarcoma tumor samples and allows for more robust statistical analysis including enrichment relative to other cancers, co-mutation and mutual exclusivity, and comparisons across the age spectrum of patients with osteosarcoma.

### Methods

### Patient and Samples

Two cohorts of patients with successful sequencing of at least one sample with a targeted next-generation sequencing (NGS) assay were included in this analysis. The “P-AYA Osteosarcoma” cohort consisted of patients equal or less than age 40 years old with at least one osteosarcoma sample sequenced with a targeted NGS assay. This cohort was identified based on a diagnosis of osteosarcoma provided by the ordering physician. A board-certified pathologist reviewed a hematoxylin and eosin (H&E) slide from each sample under light microscopy to determine tumor adequacy (approximately at least 20% tumor nuclei present) and review diagnosis. Age of 40 years of age or less was defined as the differences between testing submission year and a patient's birthyear less than or equal to 40. The “Pan-cancer” cohort consisted of all other patients in the dataset equal to or less than age 40 years old, with successful sequencing of at least one tumor sample with a targeted NGS assay. If multiple samples were present for a single patient, only the sample with the best quality, defined as samples meeting qualify control criteria regarding sequencing depth, sequencing coverage evenness, sequencing error and tumor content, was included in the final analysis. If multiple samples of comparable quality were identified for a single patient, only the most recent sample was used for analysis. Tissue of origin was determined based on the site of biopsy as submitted by the requesting physician

The P-AYA Osteosarcoma cohort was further divided into children, adolescents, and young adults (Tables 1-2). Based on prior reports on correlation between age and outcome in osteosarcoma, the three age groups were defined as follows: children included males <13 and females <12 years-of-age; adolescents included males 13 through <18, and females 12 through <18 years-of-age; young adults included all patients 18 through <40 years of age.

TABLE 1
Osteosarcoma Cohort

| | P-AYA Osteosarcoma |
|---|---|
| n | 657 |
| gender = M (%) | 393 (59.8) |
| Patient Age (median [IQR]) | 17.00 [13.00, 23.00] |
| TMBscore (median [IQR]) | 1.61 [0.81, 3.23] |

Ancestry (%)

| | |
|---|---|
| AFR | 89 (13.5) |
| AMR | 167 (25.4) |
| EAS | 28 (4.3) |
| EUR | 360 (54.8) |
| SAS | 13 (2.0) |

Age Group (%)

| | |
|---|---|
| Children | 112 (17.0) |
| Adolescents | 246 (37.4) |
| Young Adults | 299 (45.5) |

Site of tissue sample (%)

| | |
|---|---|
| Bone | 278 (42.3) |
| Lung | 188 (28.6) |
| Soft tissue | 99 (15.1) |
| Other | 92 (14.0) |

TABLE 2
Osteosarcoma Cases Compared to Pan Cancer Cohort

| | P-AYA Pan-cancer | P-AYA Osteosarcoma |
|---|---|---|
| n | 37946 | 657 |
| gender = M (%) | 16449 (43.3) | 393 (59.8) |
| Patient Age | 32.00 [21.00, 37.00] | 17.00 [13.00, 23.00] |
| (median [IQR]) | | |
| TMBscore | 1.61 [0.81, 3.48] | 1.61 [0.81, 3.23] |
| (median [IQR]) | | |

Ancestry (%)

| | | |
|---|---|---|
| African | 4952 (13.1) | 89 (13.5) |
| Admixed American | 7967 (21.0) | 167 (25.4) |
| East Asian | 1676 (4.4) | 28 (4.3) |
| European | 22467 (59.2) | 360 (54.8) |
| South Asian | 884 (2.3) | 13 (2.0) |

Age group (%)

| | | |
|---|---|---|
| Children | — | 112 (17.0) |
| Adolescents | — | 246 (37.4) |
| Adults | — | 299 (45.5) |

### Sequencing and Analysis

Sequencing was performed with two targeted NGS assays. The first is a targeted NGS assay of DNA for substitutions, insertions and deletions (indels), and copy number alterations (CNAs) in 406 genes and RNA sequencing of 265 genes for detecting rearrangements (hereafter referred to as targeted NGS assay 1). The second is a targeted NGS DNA sequencing assay detecting substitutions, indels, CNAs and rearrangements in 324 genes (hereafter referred to as targeted NGS assay 2).

Sequencing data were analyzed as described previously in Frampton, G. et al. Nat Biotechnol 31, 1023-1031 (2013). Only oncogenic or likely oncogenic alterations were included in the analysis. Oncogenic alterations were defined as genomic alterations that are predicted to drive tumorigenesis or inactivate tumor suppression. Amplification was defined as a copy number greater or equal to ploidy plus 4. All deletions discussed herein were two-copy deletions. One-copy deletions were excluded from the analysis.

### Statistical Analysis

Differences in sex and ethnicity between the P-AYA Osteosarcoma and Pan-cancer cohorts were assessed with Chi-square tests. TMB score was compared with one-way ANOVA. Significance was established at p-value≤0.05.

To identify genes that are altered more often in osteosarcoma than in other cancers, Fisher's Exact test was used to assess the frequency of oncogenic alterations in the P-AYA Osteosarcoma cohort compared to the pan-cancer cohort.

Genes were selected for further analysis and visualization in the oncoprint through literature review. Differences in the frequency of gene alterations in osteosarcoma by age group were assessed through Chi-squared tests. Significance was established at p value≤0.05.

Co-mutation and mutual exclusivity analysis was performed using Fisher's Exact test. Significance was established at adjusted p value≤0.05 with FDR correction for multiple hypothesis testing.

Statistical analysis was performed using R version 4.2.2.

### Results

### Patients and Samples

A total of 657 pediatric and young adult (<40-years-old) patients with osteosarcoma and successful sequencing with targeted NGS assay 1 were identified and included in the P-AYA Osteosarcoma cohort (Table 1). There were 112 children, 246 adolescents, and 299 young adults. Compared to the pan-cancer cohort, the P-AYA Osteosarcoma cohort included a higher proportion of male patients (59.8% vs. 43.3%; p=<0.001) and did not significantly differ in the distribution of patients from different ancestries (Table 2).

Osteosarcoma samples were most frequently obtained from bone (42%) or lung (29%), with the remaining third from soft tissue (14%) or other (15%) sites. In the P-AYA Osteosarcoma cohort, 90 patients (14%) had multiple samples.

### Specific Genes are More Commonly Altered in Osteosarcoma Compared to Other Cancers

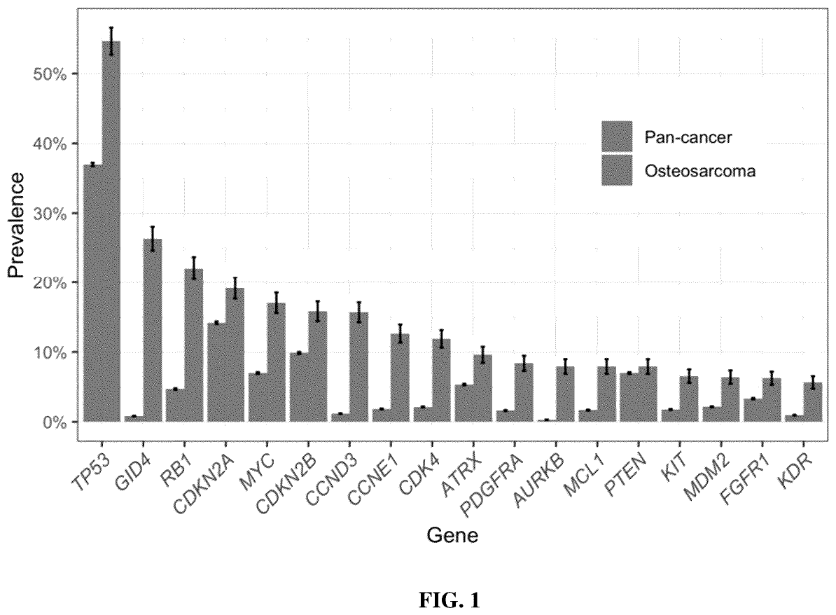

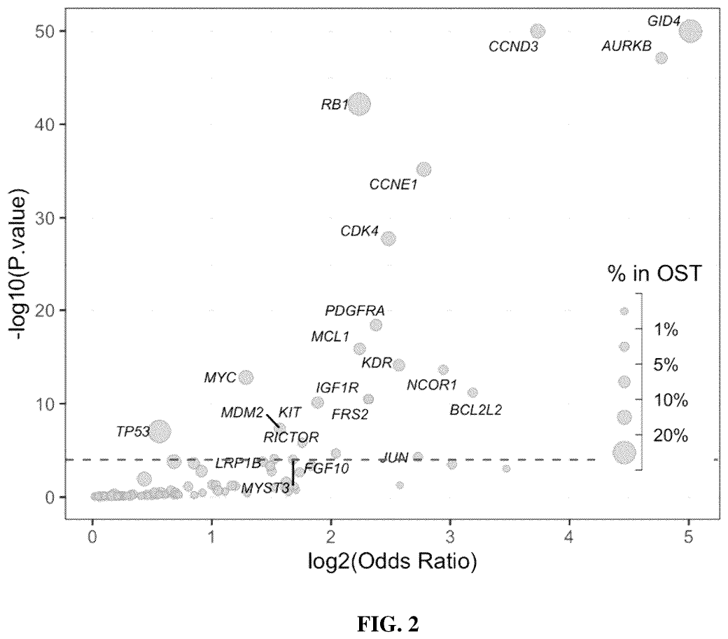

Genes that were most significantly altered in the P-AYA Osteosarcoma cohort compared to the P-AYA pan-cancer cohort were TP53 (54.6% vs 37%; OR=1.8), MYC (17% vs 7%; OR=3.6), RB1 (22.1% vs 4.7%; OR=9.4), CDK4 (11.9% vs 2.1%; OR=12), MDM2 (6.4% vs 2.1%; OR=4.8), CCNE1 (12.6% vs 1.8%; OR=16.1), KIT (6.5% vs 1.8%; OR=6.6), MCL1 (7.9% vs 1.7%; OR=9.4), PDGFRA (8.4% vs 1.6%; OR=10.8), CCND3 (15.7% vs 1.2%; OR=41.9), LRP1B (3.7% vs 1.3%; OR=4.6), RICTOR (4.1% vs 1.2%; OR=5.8), GID4 (26.3% vs 0.8%; OR=150.7), FRS2 (4.9% vs 1%; OR=10.1), IGF1R (4.9% vs 1%; OR=10.1), KDR (5.6% vs 0.9%; OR=13.1), MYST3 (3% vs 0.9%; OR=5.4), FGF10 (2.6% vs 0.6%; OR=7.7), NCOR1 (4.4% vs 0.6%; OR=18.9), AURKB (7.9% vs 0.3%; OR=118), BCL2L2 (3.2% vs 0.4%; OR=24.3), JUN (1.5% vs 0.2%; OR=15.3) (FIG. 1) with FDR adjusted p value<1e-4. One gene identified by this analysis not previously noted as recurrently altered in osteosarcoma was BCL2L2 3.2% vs 0.4%; OR=24.3; p<1e-4) (FIG. 2). TMB was 1.61 in osteosarcoma and was the same as for other cancers.

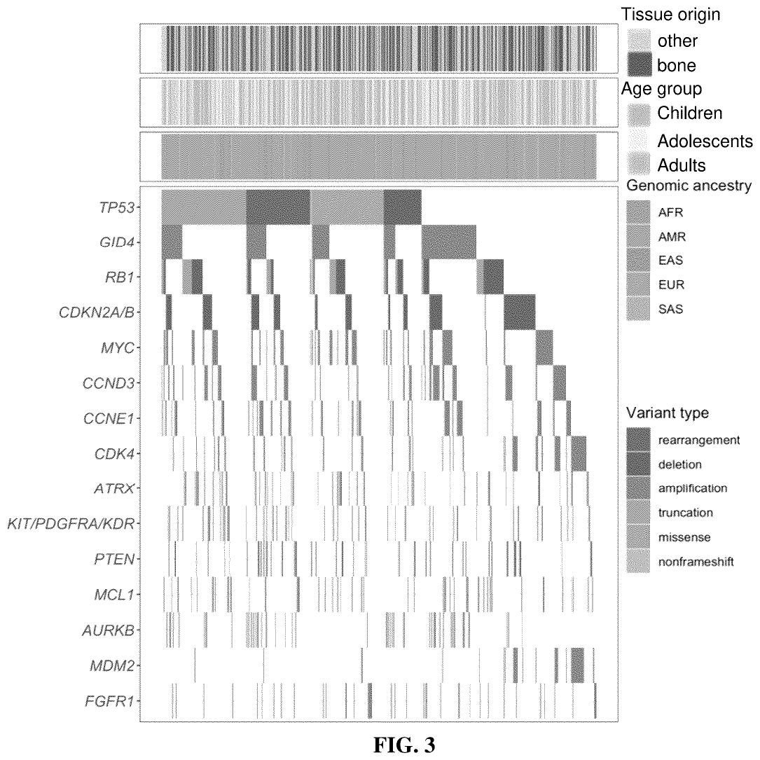

The most frequently altered gene in osteosarcoma was TP53, which was altered in 55% of samples. While TP53 is also commonly altered across different cancer types, TP53 alterations were enriched in osteosarcoma compared to other cancers (54.6% vs 37%; OR=1.8; p<1e-4) The most common types of TP53 alterations in osteosarcoma were truncations (n=117, 18%), followed by missense mutations (n=99, 15%), rearrangements (n=88, 13%), deletions (n=52, 8%) and non-frameshift mutations (n=3, 0.5%) (FIG. 3).

The most frequent gene amplifications in osteosarcoma were in GID4, MYC, CCND3, CCNE1, CDK4, AURKB, MCL1, MDM2, FGFR1 and a co-amplified section on chromosome 4q12 containing KIT/PDGFRA/KDR (FIGS. 1-2). The most frequently observed homozygous deletions, in addition to TP53, were in RB1, CDK2NA/B, ATRX and PTEN (FIG. 3). Though mutations are uncommon in osteosarcoma enriched genes overall, inactivating mutations were observed in RB1 and PTEN, and activating mutation were observed MCL1, FGFR1 and PDGFRA. Rearrangements were seen in RB1, ATRX, and PTEN (all inactivating), as well as FGFR1. Two novel fusions with preserved FGFR1 tyrosine kinase domain were identified, FGFR1::MTSS1 and TACC1::FGFR1. A large duplication in MYC that may function similarly to MYC amplification was observed in 1 cases and was characterized as a rearrangement.

### Frequency of Certain Gene Alterations Varied by Age

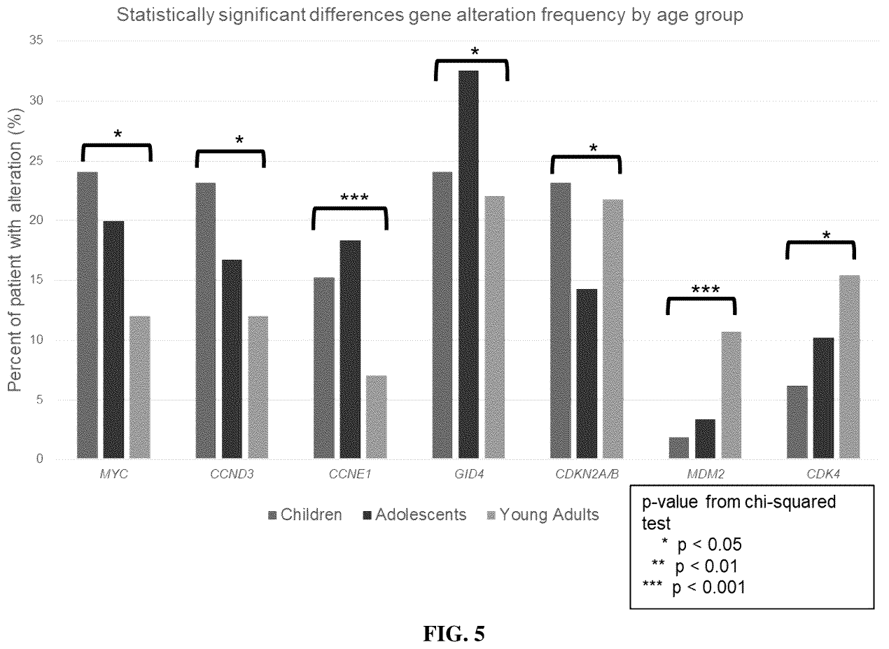

Common osteosarcoma gene alterations differ significantly by age group with seven of the 15 most frequently altered genes in osteosarcoma tested for variation with ages having a statistically significant different frequency in a three-way comparison (FIG. 5). CDK4 amplification and MDM2 amplification were most frequent in the young adult age group. CDK4 amplification occurred in 15% of tumors from young adults versus 10% in adolescents and 6% in children (p=0.022). MDM2 amplification occurred in 11% of tumors from young adults versus 3% in adolescents and 2% in children (p<0.001).

In contrast, CCNE1 amplification was least common in young adult patients with only 7% of osteosarcomas having this alteration in young adults versus 15% of children and 18% of adolescents (p<0.001). Osteosarcomas in younger patients more frequently had a MYC amplification (24% in children and 20% in adolescents compared to 12% in adults; p=0.005) and CCND3 amplification (23% in children and 17% in adolescents compared to 12% in adults; p=0.018).

Alterations with significant age variation that were most common in adolescents were GID4 amplifications (33% in adolescents compared to 24% in children and 22% in adults; p=0.019) and CCNE1 amplification (18% in adolescents compared to 15% in children and 7% in adults; p<0.001). In summary, in childhood (female <12, male <13) osteosarcomas are characterized by MYC and CCND3 amplification while MDM2 and CDK4 amplification is seen mostly in young adult (age ≥18) osteosarcoma. Osteosarcoma in adolescents commonly have GID4, CCNE1, MYC, and CCND3 amplification.

### Co-Occurrence and Mutual Exclusivity

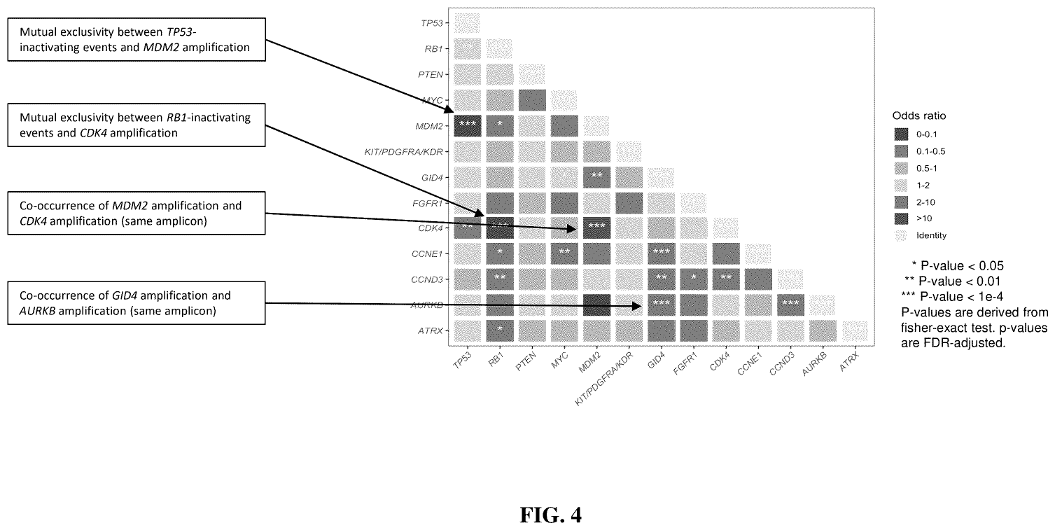

Co-occurrence and mutual exclusivity analysis was conducted on selected pairs of genes. RB1 inactivating events and CDK4, CCND3, or CCNE1 amplifications were mutually exclusive, suggesting very little overlap between alterations in genes involved in the cell cycle in osteosarcoma (FIG. 4). As previously reported [10], mutual exclusivity of MDM2 amplification and inactivating events in TP53 was observed (OR=0.08, p=4.28E-10). Amplification of KIT, PDGFRA, and KDR co-occurs due to a co-amplicon on chromosome 4 as these genes are within 896 kbp of each other. There was also co-occurrence of two genes on chromosome 17, AURKB and GID4 (OR=8.47, p=1.87E-12) despite these being 9.8 mbp distance apart from each other.

### Chromosome Arm-Level Changes

Analysis of arm-level chromosome gains and losses in osteosarcoma samples demonstrated frequent gain of chromosome 8q (50.1%) and very rare 8q loss (3.2%). There was also frequent loss of chromosome 10q (52.1%) and very rare 10q gain (0.9%).

### DISCUSSION

In this study, the genomic landscape of osteosarcoma tumor samples from 657 children, adolescents, and young adults sequenced on a single targeted platform were characterized. To date, this represents the largest number of osteosarcoma tumor samples in a single study and builds upon prior in-depth research on smaller cohorts [5-14, 19, 20]. This enables, with statistical robustness, identification of biological subtypes including differences in gene alteration frequency across the age spectrum and mutually exclusive events in DNA repair and cell cycle genes.

The most frequent alterations observed in the present study were similar to those described in prior reports [9, 10, 12-14] and included amplifications in MYC, CCND3, CCNE1, CDK4, PDGFRA, AURKB, MDM2, and FGFR1 as well as inactivating alterations in TP53, CDK2NA/B, RB1, ATRX and PTEN. Furthermore, many genes with alterations commonly reported in osteosarcoma, including RB1, AURKB, CCND3, CCNE1, and CDK4 were altered at a significantly higher frequency in osteosarcoma relative to other cancers. Of note, GID4 was the second-most frequently altered gene after TP53, was altered at a significantly higher frequency in osteosarcoma compared to other cancers and was significantly more frequent in the adolescent cohort. GID4, which lied on chromosome 17p11.2 has previously been reported to be amplified in osteosarcoma [20, 21], with a frequency of about 18%, but does not always rank among the most frequently altered genes in other reports. Amplifications of other genes at 17p11.2 have previously been noted in osteosarcoma, including NCOR1, which has been reported with a frequency of 10-20% [10, 19, 20, 22] and was significantly co-amplified with other genes at the 17p12-11.2 locus [10, 19]. Future studies that incorporate whole-genome sequencing and linked outcome data will be helpful in elucidating the potential biological and clinic relevance of CNVs at the 17p11.2 locus.

BCL2L2 alterations were significantly enriched in osteosarcoma relative to other cancers and have not previously reported as enriched in osteosarcoma. BCL2L2 encodes the protein BCL-w, which is an anti-apoptotic member of the BCL-2 family [23]. In human mesenchymal stem cells, it has been demonstrated that BCL2L2 expression prevents osteogenic differentiation [24]. The BCL2L2 transcript is a target of a multiple miRNAs which are differentially expressed in osteosarcoma [25-27]. MiR-422a and miR-133b decrease BCL2L2 expression and promote apoptosis in osteosarcoma cells [25, 26], while miR-196a increases BCL2L2 expression and promotes survival of osteosarcoma cells [27]. Future studies may investigate whether BCL2L2 amplification impacts prognosis or therapeutic sensitivities.

Comparison of gene alteration frequencies by age group revealed that MYC, CCND3, and CCNE1 amplifications were more common in younger patients, while CDK4 and MDM2 amplifications were more common in older age groups. This is largely consistent with the findings of a recent study demonstrating more frequent CNV in CCND3, AURKB, CCNE1, GID4, and MYC in P-AYA (defined as age ≤30) osteosarcomas, and more frequent MDM2, CDKN2A/B, and FRS2 alterations in adult osteosarcomas [21]. This study builds upon these findings by highlighting that differences in alteration frequencies exist by age even within the P-AYA population. Recent epidemiological studies have shown that the youngest patients with osteosarcoma have unique epidemiological features compared to older patients, including higher overall 5-year survival and similar incidence and survival rates between sexes, suggesting possible differences in the underlying etiology of osteosarcoma among pre-pubertal patients [3]. The interaction of age and tumor biology is further highlighted by the finding that up to a quarter of the youngest patients with osteosarcoma carry a pathogenic/likely pathogenic germline variant in an autosomal-dominant cancer-susceptibility gene [28]. Thus, achieving greater granularity in age cohorts within the P-AYA age group may provide important information in defining biological subtypes.

Additional studies that include linked genomic and clinical data from large cohorts of patients with osteosarcoma across the age spectrum are needed to uncover the potential clinical implications of age-related differences in tumor biology. For example, it has been demonstrated that MYC amplification is associated with poorer outcomes among patients with osteosarcoma [12, 14]. It would be important to consider the frequency of MYC amplification in different age groups if this observation is validated in prospective clinical trials, and ultimately considered in risk stratification.

Taken together, analysis of gene alteration frequencies by age group and mutual exclusivity analysis may allow for insights into important pathways in osteosarcoma. Mutual exclusivity of alterations in RB1, CDK4, CCND3 and CCNE1 was observed across all osteosarcoma samples. RB1 was mutated at the same frequency across age groups; however, CDK4, CCND3 and CCNE1 were altered at different frequencies in the three age cohorts (the former being more common in older patients, the latter two more common in younger patients). This may suggest that the mechanism of cell cycle dysregulation in osteosarcoma is associated with age. Mutual exclusivity was also found between MDM2 and TP53 alterations. MDM2 amplification occurred significantly more in the young adult group relative to younger patients. Furthermore, MDM2 and CDK4 co-amplification at 12q13-15 has been well-described in various cancers including neuroblastoma [29], dedifferentiated liposarcoma [30, 31] and osteosarcoma [32]. The potential clinical implications of these age-related differences in tumor biology warrants further investigation.

In addition to identification of biological subtypes, analysis of this large cohort helps validate the relative frequencies of genetic alterations in P-AYA patients with osteosarcoma, which may aid in the design of clinical trials for targeted therapies from which young patients with osteosarcoma are likely to benefit. Multi-targeted tyrosine kinase inhibitors (mTKIs) have been prioritized for frontline evaluation in osteosarcoma [33] due to demonstrated single-agent activity of agents including regorafenib [34, 35], sorafenib [36], lenvatinib [37], and cabozantinib [38] in phase II clinical trials. A phase II/III trial to test the addition of cabozantinib to standard chemotherapy with methotrexate, doxorubicin, and cisplatin (MAP) in patients with newly diagnosed osteosarcoma is ongoing (COG AOST2032, National Clinical Trial Identifier: NCT05691478). Genomic information was not provided in the phase II study of single agent cabozantinib [38]; however, it would be interesting to compare responders and non-responders in ongoing trials to determine whether there are genomic biomarkers predictive of response. Prior studies have demonstrated 4q12 amplification including KIT, KDR and PDGFRA at a frequency of about 15-20% [10]. A slightly lower frequency in the cohort was noted, up to about 10% in patients <18 years old. Nevertheless, this represents a relatively large proportion of patients for whom targeted therapy with mTKIs targeted at KIT, KDR, and/or PDGFRA might be feasible. For example, a pediatric investigation plan has been proposed for avapritinib, a highly selective KIT and PDGFRA inhibitor, which would include solid tumors dependent on KIT or PDGFRA signaling [39].

In keeping with prior studies, ATRX was also among the most frequently altered genes in osteosarcoma samples, with a frequency of about 10% [10]. ATRX plays a role in diverse biological pathways including epigenetic modification, alternative lengthening of telomeres, and maintenance of genome integrity [40]. In vitro and in vivo models of osteosarcoma have shown that loss of ATRX expression leads to increased tumor migration, invasion, and metastasis, which may be mediated through effects on ETS family transcription factor binding, NF-kB signaling, and integrin expression [41]. In samples from patients with osteosarcoma, lower expression of ATRX was significantly correlated with longer normalized telomere length, with the longest telomere lengths occurring in tumors that harbored alterations in both TP53 and ATRX, [11]. ATRX-deficient cells have increased sensitivity to several therapies including ATM inhibitors, the topoisomerase I inhibitor irinotecan (alone or combined with the PARP inhibitor, Olaparib), RNA polymerase I inhibitors, synthetic inhibitors of FANCM and BTR, which are involved in restricting telomeric replication stress, and WEE1 inhibitors [40]. Interestingly, a recent screen for lethal compounds in ATRX-deficient high-grade glioma cells demonstrated increased sensitivity to several mTKIs and a specific PDGFR inhibitors [42], thus, ATRX deficiency might also be investigated as predictor of response to mTKIs.

This study relied on existing sequencing data generated in a commercial laboratory, which offers several research benefits, including access to large numbers of patient samples analyzed with a uniform platform. However, this approach also has several limitations, including a lack of linked clinical and genomic data, particularly outcome data, which is essential to confirm the clinical relevance of biological subtypes. Future studies using linked datasets may help determine whether distinctions observed in this analysis, such as the mechanisms of TP53 inactivation or cell cycle dysregulation, have an impact on therapy and survival. While targeted sequencing is a widely accessible option, there are several observations for which more in-depth analysis with whole genome sequencing (WGS), paired samples, and multi-omics analysis can help provide further information. For example, WGS may enrich understanding of the relevance of amplifications such as those at the 17p11.2 region, and other structural variants. Paired somatic and germline samples may help uncover links between germline predisposition and age-related differences in tumor biology. Furthermore, analysis of multiple tumor samples from an individual patient, available in 14% of cases (n=90) within the P-AYA Osteosarcoma cohort, may reveal important patterns in tumor evolution. Multi-omics may provide further information about how genomic changes alter cell biology and ultimately prognosis. For example, MYC amplification was associated with worse prognosis overall in a recent multi-omics study, but this effect was modified by homologous recombination (HR) status, with HR-proficient cells having higher MYC expression and worse prognosis [12].

In summary, this study describes the genomic landscape of osteosarcoma using the largest number of osteosarcoma samples to date, with statistically robust assessment of mutational patterns across the age spectrum of patients with osteosarcoma. The assembly of large datasets that include not only targeted sequencing data, but also whole-genome, multi-omic, and multiple-sample analysis linked to clinical outcome data are a critical next step in deepening understanding of the genomic landscape of osteosarcoma and translating this knowledge into improved treatment strategies for patients.

### REFERENCES

1. NCCR Explorer: An interactive website for NCCR cancer statistics.
2. Mirabello, L., R. J. Troisi, and S. A. Savage, Osteosarcoma incidence and survival rates from 1973 to 2004: datafrom the Surveillance, Epidemiology, and End Results Program. Cancer, 2009. 115(7): p. 1531-43.
3. Cole, S., et al., Osteosarcoma: A Surveillance, Epidemiology, and End Results program-based analysis from 1975 to 2017. Cancer, 2022. 128(11): p. 2107-2118.
4. Meltzer, P. S. and L. J. Helman, New Horizons in the Treatment of Osteosarcoma. N Engl J Med, 2021. 385(22): p. 2066-2076.
5. Chen, X., et al., Recurrent somatic structural variations contribute to tumorigenesis in pediatric osteosarcoma. Cell Rep, 2014. 7(1): p. 104-12.
6. Perry, J. A., et al., Complementary genomic approaches highlight the PI3K/mTOR pathway as a common vulnerability in osteosarcoma. Proc Natl Acad Sci USA, 2014. 111(51): p. E5564-73.
7. Kovac, M., et al., Exome sequencing of osteosarcoma reveals mutation signatures reminiscent of BRCA deficiency. Nat Commun, 2015. 6: p. 8940.
8. Behjati, S., et al., Recurrent mutation of IGF signalling genes and distinct patterns of genomic rearrangement in osteosarcoma. Nat Commun, 2017. 8: p. 15936.
9. Sayles, L. C., et al., Genome-Informed Targeted Therapy for Osteosarcoma. Cancer Discov, 2019. 9(1): p. 46-63.
10. Suehara, Y., et al., Clinical Genomic Sequencing of Pediatric and Adult Osteosarcoma Reveals Distinct Molecular Subsets with Potentially Targetable Alterations. Clin Cancer Res, 2019. 25(21): p. 6346-6356.
11. Wu, C. C., et al., Immuno-genomic landscape of osteosarcoma. Nat Commun, 2020. 11(1): p. 1008.
12. Jiang, Y., et al., Multi-omics analysis identifies osteosarcoma subtypes with distinct prognosis indicating stratified treatment. Nat Commun, 2022. 13(1): p. 7207.
13. Kinnaman, M. D., et al., Subclonal somatic copy number alterations emerge and dominate in recurrent osteosarcoma. bioRxiv, 2023.
14. Marinoff, A. E., et al., Clinical Targeted Next-Generation Panel Sequencing Reveals MYC Amplification Is a Poor Prognostic Factor in Osteosarcoma. JCO Precis Oncol, 2023. 7: p. e2200334.
15. Cortes-Ciriano, I., et al., Comprehensive analysis of chromothripsis in 2,658 human cancers using whole-genome sequencing. Nat Genet, 2020. 52(3): p. 331-341.
16. Gill, J. and R. Gorlick, Advancing therapy for osteosarcoma. Nat Rev Clin Oncol, 2021. 18(10): p. 609-624.
17. Busse, T. M., et al., Copy number alterations determined by single nucleotide polymorphism array testing in the clinical laboratory are indicative of gene fusions in pediatric cancer patients. Genes Chromosomes Cancer, 2017. 56(10): p. 730-749.
18. McConnell, L., et al., A novel next generation sequencing approach to improve sarcoma diagnosis. Mod Pathol, 2020. 33(7): p. 1350-1359.
19. Nacev, B. A., et al., Clinical sequencing of soft tissue and bone sarcomas delineates diverse genomic landscapes and potential therapeutic targets. Nat Commun, 2022. 13(1): p. 3405.
20. Xie, X., et al., A Comprehensive Understanding of the Genomic Bone Tumor Landscape: A Multicenter Prospective Study. Front Oncol, 2022. 12: p. 835004.
21. Gounder, M. M., et al., Clinical genomic profiling in the management of patients with soft tissue and bone sarcoma. Nat Commun, 2022. 13(1): p. 3406.
22. Luo, Z., et al., Integrative analysis reveals driver long non-coding RNAs in osteosarcoma. Medicine (Baltimore), 2019. 98(6): p. e14302.
23. Hartman, M. L. and M. Czyz, BCL-w: apoptotic and non-apoptotic role in health and disease. Cell Death Dis, 2020. 11(4): p. 260.
24. Tzeng, S. Y., et al., Cystamine-terminated poly(beta-amino ester)s for siRNA delivery to human mesenchymal stem cells and enhancement of osteogenic differentiation. Biomaterials, 2012. 33(32): p. 8142-51.
25. Zhang, H., et al., miR-422a inhibits osteosarcoma proliferation by targeting BCL2L2 and KRAS. Biosci Rep, 2018. 38(2).
26. Zhao, H., et al., MiR-133b is down-regulated in human osteosarcoma and inhibits osteosarcoma cells proliferation, migration and invasion, and promotes apoptosis. PLoS One, 2013. 8(12): p. e83571.
27. Shang, Y., et al., MicroRNA-196α overexpression promotes cell proliferation and inhibits cell apoptosis through PTEN/Akt/FOXO1pathway. Int J Clin Exp Pathol, 2015. 8(3): p. 2461-72.
28. Mirabello, L., et al., Frequency of Pathogenic Germline Variants in Cancer-Susceptibility Genes in Patients With Osteosarcoma. JAMA Oncol, 2020. 6(5): p. 724-734.
29. Martinez-Monleon, A., et al., Amplification of CDK4 and MDM2: a detailed study of a high—risk neuroblastoma subgroup. Sci Rep, 2022. 12(1): p. 12420.
30. Ricciotti, R. W., et al., High amplification levels of MDM2 and CDK4 correlate with poor outcome in patients with dedifferentiated liposarcoma: A cytogenomic microarray analysis of 47 cases. Cancer Genet, 2017. 218-219: p. 69-80.
31. Italiano, A., et al., HMGA2 is the partner of MDM2 in well-differentiated and dedifferentiated liposarcomas whereas CDK4 belongs to a distinct inconsistent amplicon. Int J Cancer, 2008. 122(10): p. 2233-41.
32. Yoshida, A., et al., MDM2 and CDK4 immunohistochemical coexpression in high-grade osteosarcoma: correlation with a dedifferentiated subtype. Am J Surg Pathol, 2012. 36(3): p. 423-31.
33. Whittle, S. B., et al., Charting a path for prioritization of novel agents for clinical trials in osteosarcoma: A report from the Children's Oncology Group New Agents for Osteosarcoma Task Force. Pediatr Blood Cancer, 2021. 68(9): p. e29188.
34. Davis, L. E., et al., Randomized Double-Blind Phase II Study of Regorafenib in Patients With Metastatic Osteosarcoma. J Clin Oncol, 2019. 37(16): p. 1424-1431.
35. Duffaud, F., et al., Efficacy and safety of regorafenib in adult patients with metastatic osteosarcoma: a non-comparative, randomised, double-blind, placebo-controlled, phase 2 study. Lancet Oncol, 2019. 20(1): p. 120-133.
36. Grignani, G., et al., A phase II trial of sorafenib in relapsed and unresectable high-grade osteosarcoma after failure of standard multimodal therapy: an Italian Sarcoma Group study. Ann Oncol, 2012. 23(2): p. 508-16.
37. Gaspar, N., et al., Phase I/II study of single-agent lenvatinib in children and adolescents with refractory or relapsed solid malignancies and young adults with osteosarcoma (ITCC-050)(⋆). ESMO Open, 2021. 6(5): p. 100250.
38. Italiano, A., et al., Cabozantinib inpatients with advanced Ewing sarcoma or osteosarcoma (CABONE): a multicentre, single-arm, phase 2 trial. Lancet Oncol, 2020. 21(3): p. 446-455.
39. Pearson, A. D., et al., Paediatric Strategy Forum for medicinal product development of multi—targeted kinase inhibitors in bone sarcomas: ACCELERATE in collaboration with the European Medicines Agency with participation of the Food and Drug Administration. Eur J Cancer, 2022. 173: p. 71-90.
40. Aguilera, P. and A. J. Lopez-Contreras, ATRX a guardian of chromatin. Trends Genet, 2023.
41. Bartholf DeWitt, S., et al., Loss of ATRX promotes aggressive features of osteosarcoma with increased NF-kappaB signaling and integrin binding. JCI Insight, 2022. 7(17).
42. Pladevall-Morera, D., et al., ATRX-Deficient High-Grade Glioma Cells Exhibit Increased Sensitivity to RTK and PDGFR Inhibitors. Cancers (Basel), 2022. 14(7).

1. A method of treating or delaying progression of osteosarcoma in an individual, comprising:
(a) detecting an FGFR1-MTSS1 or TACC1-FGFR1 gene fusion in one or more samples from an individual having osteosarcoma; and
(b) administering to the individual an effective amount of a treatment that comprises an anti-cancer agent.

2. The method of claim 1, wherein the anti-cancer agent comprises an FGFR1 inhibitor, tyrosine kinase inhibitor, or pan-kinase inhibitor.

3. The method of claim 1, wherein the FGFR1-MTSS1 gene fusion encodes a fusion polypeptide comprising the amino acid sequence of SEQ ID NO:1.

4. The method of claim 1, wherein the FGFR1-MTSS1 gene fusion comprises the polynucleotide sequence of SEQ ID NO:2.

5. The method of claim 1, wherein the TACC1-FGFR1 gene fusion encodes a fusion polypeptide comprising the amino acid sequence of SEQ ID NO:3.

6. The method of claim 1, wherein the TACC1-FGFR1 gene fusion comprises the polynucleotide sequence of SEQ ID NO:4.


SEQUENCE LISTING (4 sequences)

SEQ ID NO: 1 - protein, 779 aa, synthetic construct
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQDNSSSSSCLLGRLSESG

SEQ ID NO: 2 - DNA, 4554 bp, synthetic construct
  source 1..4554: mol_type = other DNA
atgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggactgtattttgagcctgaaccaatttcttccacgcccaattatttgcaacggggagaattttccagttgtgtttcatgtgaagaaaactcaagctgcctcgaccagatctttgattcctaccttcagacagaggacaacagtagtagcagcagctgccttcttggacgcctttcagaaagtggctgacatggccaccaacacacgtggtgggaccagggagattggatctgctctcaccaggatgtgcatgaggcacagaagcattgaagccaagctgaggcagttttcgagcgctttaattgattgtctgataaacccacttcaagaacagatggaagaatggaagaaagtggccaaccagctggataaagaccacgcaaaagaatataagaaagcccgccaagagataaaaaagaagtcctcggatacgctgaaactgcagaagaaagcaaaaaaagggagaggtgatatccagcctcagttggacagtgctctccaagatgtcaatgataagtatctcttattggaagaaacagaaaagcaggctgtccggaaggctttgattgaagaacgtggccgattctgtaccttcatctctatgctgcggccagtgattgaagaagaaatctcaatgctaggggaaataacccaccttcagaccatctcggaagatctaaaaagcctgaccatggaccctcacaaactgccctcctcaagtgaacaggtgattctggacttgaaaggttctgattacagctggtcgtatcagacgccaccctcttcccccagcaccaccatgtccagaaagtccagtgtctgcagcagcctgaacagtgtcaacagcagtgactcccggtccagcggctcccactcgcattcccccagctcacattaccgctaccgcagctccaacctggcccagcaggctcctgtgaggctgtccagcgtgtcctcccatgactcaggattcatatcccaggatgccttccagtccaagtcaccatcccccatgccgccagaggcccccaaccagttgtctaacgggttttctcactatagtttatcaagtgagtcccacgtggggcccacgggtgcaggccttttccctcattgcctgcctgcctcccgcctgctccctcgggtcacctctgtccaccttccagactacgctcattattacaccattgggcccggcatgttcccgtcatctcagatccctagctggaaggactgggctaagcctgggccctatgaccagcctctggtgaacaccctgcagcgccgcaaagagaagcgagaaccggaccccaacgggggaggacccactaccgccagcggcccacctgcagcagctgaggaggctcagagaccacggagcatgactgtatcggctgccaccaggcctggtgaggagatggaggcttgtgaggagctggccctggccctgtctcggggcctgcagctggacacccagaggagcagccgggactcgcttcagtgctccagcggctacagcacccagacaaccaccccctgctgctctgaggacaccatcccttcccaagtttcagattatgattatttctctgtaagtggtgaccaggaggcagatcagcaggagttcgacaagtcctccaccattccaagaaacagcgacatcagccagtcctaccgacggatgttccaagccaagcgtccagcctcaactgctggcctccccaccaccctgggacctgctatggtcactccaggggttgcaactatccgacggaccccttccaccaagccttctgtccgccggggaaccattggagctggtcccatccccatcaagacacccgtgatccctgtcaagaccccaaccgtcccagacctcccaggggtgttgccagcccctccagatgggccagaagagcggggggagcacagccctgagtcgccatctgtgggtgagggcccccaaggtgtcaccagcatgccctcctcaatgtggagcggccaagcttccgttaaccctccacttccaggcccgaagcccagtatccctgaggagcacagacaggcaattccagaaagtgaagctgaagaccaggaacgggaacccccaagtgccactgtctccccaggccagattccagagagtgaccctgcagacctgagcccaagggatactccacaaggagaagacatgctgaacgccatccgaaggggcgtgaaactgaagaagaccacgacaaacgatcgctcagcccctcgcttttcttag

SEQ ID NO: 3 - protein, 873 aa, synthetic construct
MAFSPWQILSPVQWAKWTWSAVRGGAAGEDEAGGPEGDPEEEDSQAETKSLSFMWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 4 - DNA, 2758 bp, synthetic construct
  source 1..2758: mol_type = other DNA
atggcgttcagcccgtggcagatcctgtcccccgtgcagtgggcgaaatggacgtggtctgcggtacgcggcggggccgccggcgaggacgaggctggcgggcccgagggcgaccccgaggaggaggattcgcaagccgagaccaaatccttgagtttcaggactgtattttgagcctgaaccaatttcttccacgcccaattatttgcaacggggagaattttccagttgtgtttcatgtgaagaaaactcaagctgcctcgaccagatctttgattcctaccttcagacagagatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctga